(19) United States
(12) Patent Application Publication
(10) Pub. No.: US 2022/0375623 A1
(43) Pub. Date: Nov. 24, 2022

(54) SYSTEM AND METHOD FOR IDENTIFICATION OF ASTHMA TRIGGERING CONDITIONS BASED ON MEDICAMENT DEVICE MONITORING FOR A PATENT

(71) Applicant: RECIPROCAL LABS CORPORATION (D/B/A PROPELLER HEALTH), Madison, WI (US)

(72) Inventor: NICHOLAS JOHN HIRONS, Madison, WI (US)

(21) Appl. No.: 17/773,469

(22) PCT Filed: Oct. 23, 2020
(86) PCT No.: PCT/US2020/057196

§ 371 Date: Apr. 29, 2022

Related U.S. Application Data
(60) Provisional application No. 62/928,937, filed on Oct. 31, 2019.

(51) Int. Cl. G16H 50/70; G16H 20/10

(52) U.S. Cl. CPC G16H 50/70; G16H 20/10

(57) ABSTRACT

A method for determining risk of triggering a respiratory ailment in comparison to a general patient population is disclosed. Data on activation of medicament devices to deliver respiration medicament to a population of patients is collected. The activation data and patient contextual parameter data related to each activation event is stored. A data analysis module determines the occurrence of rescue events based on the collected activation data, and determines a coefficient for a contextual parameter as a triggering event for a primary patient based on the correlation of the contextual parameter and the rescue event. The data analysis module provides a comparison of the coefficient for the at least one contextual parameter for the primary patient and the distributions of coefficients of the triggering event for the population of patients based on the correlation of the contextual parameter and the rescue events for the population of patients.

### PRIORITY CLAIM

This application claims the benefit of, and priority to, to U.S. Provisional Patent Application No. 62/928,937, filed on Oct. 31, 2019, which is hereby incorporated by reference herein in its entirety.

### TECHNICAL FIELD

The present disclosure relates generally to methods of improving treatment for patients with potential respiratory ailments, and more specifically to determining a patient's causes of asthma-related rescue events relative to the susceptibility of a general patient population.

### BACKGROUND

Asthma remains a significant and costly public health problem. In the United States, more than 22 million people have the disease. Worldwide, the World Health Organization estimates the population with asthma may be 300 million, and predicts that it will rise to 400 million by 2025.

Despite the development of new medications, rates of hospitalizations and emergency room visits have not declined. Each year in the United States the disease causes approximately 2 million emergency department visits, 500,000 hospitalizations, and 5,000 deaths. In addition, asthma is responsible for an estimated 15 million missed days of school, and 12 million days of work. Total annual costs to US health insurers and employers are greater than $18 billion.

The majority of these exacerbations could be prevented with currently available treatments, however, only 1 in 5 asthmatics has the disease under control. Such treatments often rely on identifying a triggering condition of a respiratory condition such as asthma and properly administering treatment such as a medicament. One mechanism for a patient to self administer a medicament is an inhaler. When a trigger event occurs, a patient may administer the medicament via a puff from the inhaler.

Newly revised national guidelines urge doctors to more closely monitor whether treatment is controlling everyday symptoms and improving quality of life. An increasing number of physicians have begun to use periodic, written questionnaires (such as the Asthma Control Test) to monitor patients and their conditions. These instruments require patients to accurately recall and report the symptoms, frequency of symptoms, inhaler usage, and activity level and restriction over some period of time (usually two to four weeks). As a result, these questionnaires are subject to error introduced by biases (recall), different interpretations of symptoms, and behaviors (non-adherence), and only provide information at the time they are used.

One recent approach has been determining triggering events through statistical analysis based on individual patient outcomes. Such triggering events are typically associated with puffs from an inhaler of medicament. Attempts have been made to analyze when such events and thus puffs may occur. However, predicting puffs is a very noisy and error-prone problem. Several issues make such analysis is difficult. First, high error rates occur, even under idealized conditions. There are many candidate “trigger” variables. They are often correlated. When variables are portioned into buckets based on whether they are greater than or less than certain thresholds (e.g. “temperature above 80”, “temperature above 90” etc.), even more possible variables that may be correlated are created. Conducting multiple hypothesis tests, especially with correlated treatment variables, leads to a much greater chance of false positives of a “significant” relationship to a variable even if the correlation is due to random chance.

Even higher error rates occur when the event occurs infrequently (such as less than 30% of the time). The event may be whether or not there was a puff that day, or whether or not the puff count was above some baseline. Simulations indicate that sequential probability ratio testing can have both high false positive rates and high false negative rates when the event occurs at the lower rates actually seen with patients. Adjusting and correcting for these factors is challenging in a practical setting.

Second, current statistical analysis methods require an impractically long time horizon before a trigger condition may be identified because they largely rely on data from the primary patient. Unless the relationship between a variable and an outcome is very strong, it can take a very long time to “discover” if someone is sensitive to an environmental trigger or not. For example, in a simulated situation where an increase over baseline occurred at a 50% greater rate for a patient when temperatures were above 80 degrees, the tests often still had not identified the relationship after 18 months of data. This is further exacerbated by the fact that in order to adequately control for the infrequency of certain events, the time horizon to confirm the identification of a trigger condition becomes even longer.

Third, it is difficult to communicate potential useful triggering information to patients to comply with current and future regulations. Issues such as statistical significance and expression of confidence in statistical prediction are difficult to explain to a patient. Errors in the approach may also impact compliance with regulations.

Statistical significance is challenging to understand. Determining such significance with multiple correlated variables is even more challenging. Determining the statistical significance on a sequential basis is even more challenging. Adjusting for these issues and still having practical insights on triggering conditions to communicate to patients via an application within a reasonable timeframe currently is all but impossible.

There is a need for a system that allows for determination of a triggering event from environmental data for a primary patient in comparison to the distribution of the triggering event probability from a large population of patients. There is also a need for providing triggering event analysis in a relatively short period of time. There also is a need to provide statistically based trigger analysis in a form that is relatively easy to understand for a patient.

### SUMMARY

The disclosed respiratory ailment monitoring system provides a method and system to more accurately predict trigger conditions and communicate such conditions to patients. The system collects sensor data from treatment devices such as inhalers, patient demographic and physiological data and environmental data in relation to a patient population with respiratory ailments. The data is collected and organized into a form where each row of data is a patient day, with environmental exposures and inhaler usage in each column. The analysis gathers frequentist statistics to relative sensitivity compared to every other medicament inhaler patient in the patient population. The resulting analysis is social, dynamic, and engaging because it makes comparisons relative to other patients. Thus, the resulting analysis is easy to understand as a patient.

One disclosed example is a system to determine conditions that trigger a respiratory ailment. A communication interface collects data on activation of respiration medicament devices to deliver respiration medicament to patients in a population of patients. A storage device stores the activation data and patient contextual parameter data related to each activation event for the population of patients. A data analysis module is operable to access the activation data and the contextual parameter data for a primary patient in the population of patients over a certain period of time. The data analysis module determines the occurrence of rescue events based on the collected activation data, and determines a coefficient for at least one contextual parameter as a triggering event for the primary patient based on the correlation of the contextual parameter and the rescue events. The data analysis module provides a comparison of the coefficient for the at least one contextual parameter for the primary patient and the distributions of coefficients of the triggering event for the population of patients based on the correlation of the contextual parameter and the rescue events for the population of patients.

In another implementation of the disclosed example system, the coefficient is determined partially based on the contextual parameter data and the occurrence of rescue events based on the collected activation data for at least one secondary patient similar to primary patient of the population of patients for the contextual parameter of the primary patient. In another implementation, the coefficient is determined based on regression analysis of the contextual parameter in relation to the collected activation data optimized by at least one hyperparameter. In another implementation, the regression analysis is performed after a first predetermined period of time for collected activation data and contextual data. In another implementation, the coefficients of the population of patients are selected based on regression analysis performed after the first predetermined period of time for each of the patients in the population of patients. In another implementation, the at least one hyperparameter is tuned based on the collected contextual parameter data for the population of patients over a second predetermined period of time. In another implementation, the respiratory ailment is asthma. In another implementation, the at least one contextual parameter includes one of air pollutant condition or weather condition. In another implementation, the pollutant condition is one of air quality index, ozone molecules (O3), nitrogen dioxide molecules (NO2), sulfur dioxide molecules (SO2), particulate matter, 2.5 micrometers or less (PM2.5), particulate matter, 10 micrometers or less (PM10). In another implementation, the weather condition is one of temperature, humidity, wind speed, wind direction, station pressure, and visibility. In another implementation, the data analysis module determines multiple trigger conditions including the determined trigger condition. The data analysis module assigns a coefficient for each of the multiple trigger conditions. The data analysis module compares the coefficients to a distribution of coefficients for each of the multiple trigger conditions from the population of patients. In another implementation, the system includes an output application. The output application displays selected trigger conditions from the multiple trigger conditions based on whether the coefficient for the primary patient is in a certain percentile of the distribution of the coefficients for the population of patients. In another implementation, the output application assigns a descriptive label correlated with an overall risk factor for the primary patient. In another implementation, the system includes a display displaying the descriptive label to the primary patient. In another implementation, the system includes a mobile device coupled to the data analysis module. In another implementation, the data analysis module alerts the primary patient if the coefficient is in a high percentile of the distribution of coefficients of the population of patients.

Another disclosed example is a method to evaluate a triggering event for a respiratory ailment of a primary patient. Usage data from activation of treatment devices is collected from a population of patients from a communication interface. Contextual parameter data corresponding to the population of patients is collected from the communication interface. The collected usage data and contextual parameter data is stored in a storage device. A coefficient for a triggering event is determined from the contextual parameter and usage data for each patient of the population of patients. A coefficient for the triggering event for the primary patient based on data relating to the contextual data and usage data for the primary patient is determined. A comparison of the coefficient of the primary patient is provided in relation to a distribution of the coefficients for the population of patients.

In another implementation of the disclosed example method, activation data is collected for at least one secondary patient similar to primary patient of the population of patients for the contextual parameter of the primary patient. The coefficient is determined partially based on the contextual parameter data and the occurrence of rescue events based on the at least one secondary patient. In another implementation, the coefficient is determined based on regression analysis of the contextual parameter in relation to the collected activation data optimized by at least one hyperparameter. In another implementation, the regression analysis is performed after a first predetermined period of time for collected activation data and contextual data. In another implementation, the coefficients of the population of patients are selected based on regression analysis performed after the first predetermined period of time for each patient in the population of patients. In another implementation, the at least one hyperparameter is tuned based on the collected contextual parameter data for the population of patients over a second predetermined period of time. In another implementation, the respiratory ailment is asthma. In another implementation, the at least one contextual parameter includes one of air pollutant condition or weather condition. In another implementation, the pollutant condition is one of air quality index, ozone molecules (O3), nitrogen dioxide molecules (NO2), sulfur dioxide molecules (SO2), particulate matter, 2.5 micrometers or less (PM2.5), or particulate matter, 10 micrometers or less (PM10). In another implementation, the weather condition is one of temperature, humidity, wind speed, wind direction, station pressure, and visibility. In another implementation, the method includes determining multiple trigger conditions including the determined trigger condition. The method includes assigning a coefficient for each of the multiple trigger conditions. The method includes comparing the coefficients to a distribution of coefficients for each of the multiple trigger conditions from the population of patients. In another implementation, the method includes displaying selected trigger conditions from the multiple trigger conditions based on whether the coefficient for the primary patient is in a certain percentile of the distribution of the coefficients for the population of patients. In another implementation, the method includes assigning a descriptive label correlated with an overall risk factor for the primary patient. In another implementation, the method includes displaying the descriptive label to the primary patient. In another implementation, the display of the descriptive label and selected triggering conditions are performed on a display of a mobile device. In another implementation, the method includes alerting the primary patient if the coefficient is in a high percentile of the distribution of coefficients of the population of patients.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a respiratory ailment analytics system for monitoring accurate, real-time medicament device usage, performing analytics on that data, and providing rescue event risk notifications;

FIG. 2 is a high-level block diagram illustrating an example of a computing device used in either as a client device, application server, and/or database server;

FIG. 3A shows a dashboard of a client application that allows a user to interact with an asthma analytics system;

FIG. 3B shows an example card displayed within the dashboard of the client application;

FIG. 4 is a flowchart for detecting a rescue medication event by an asthma analytics system for a primary patient;

FIG. 5 is a block diagram illustrating the modules within the data analysis module to generate a primary patient coefficient compared to the distribution of coefficients from a patient population;

FIG. 6A is a block diagram of the process of discretization of the discretization module in FIG. 5;

FIG. 6B is a block diagram of the patient similarity module in FIG. 5;

FIG. 6C is a block diagram of the regression module in FIG. 5;

FIG. 6D is a block diagram of the relative ranking module in FIG. 5;

FIG. 7 is a screen image of an interface of a patient application that shows presentation of selected primary patient data based on comparison to data distribution from a general patient population; and

FIG. 8 is a flowchart for identifying triggers for individual patients and producing labels for the triggers in comparison to a general population of patients, according to one embodiment.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that collects large population data for comparison to a primary patient to present trigger notifications for respiratory disorders such as asthma. The system collects sensor data from treatment devices such as inhalers to determine adherence and potential triggers of respiratory events based on usage data of the inhaler. The collected data is combined with patient demographic and physiological data as well as relevant environmental data. The analysis gathers frequentist statistics to relative sensitivity of an environmental parameter as a trigger event compared to every other medicament inhaler patient in the patient population. The resulting analysis is social, dynamic, and engaging because it makes comparisons of a triggering condition relative to that condition affecting other patients in the general patient population. Thus, the resulting analysis relating to one or more triggering conditions is easy to understand as a patient.

The system recognizes that there is no absolute knowledge as to what exactly triggers asthma in a current patient and the confidence of any potential triggering condition cannot be determined, especially with an often very limited amount of daily data. The disclosed system incorporates this initial uncertainty and the expectation that relationships between conditions and triggering events are expected to change over time as more data is gathered. The disclosed method allows some insights to be provided early in the analysis, but also continuously, without making any claims of exact knowledge.

At fixed milestones on the platform (for example, every 30 days), a regression analysis is performed on the entire daily history for each patient in the patient population. Coefficients for each contextual parameter, such as temperature, humidity, particle counts and air pollution, are determined. Each coefficient is ranked as a percentile of every other patient in the patient population up to that milestone. If the coefficient is a high enough rank, the patient may be informed that they have above average sensitivity to that environmental parameter compared to other patients in the population. When the variable condition occurs in the future, the user may be informed, and the forecast output of a triggering condition may be adjusted.

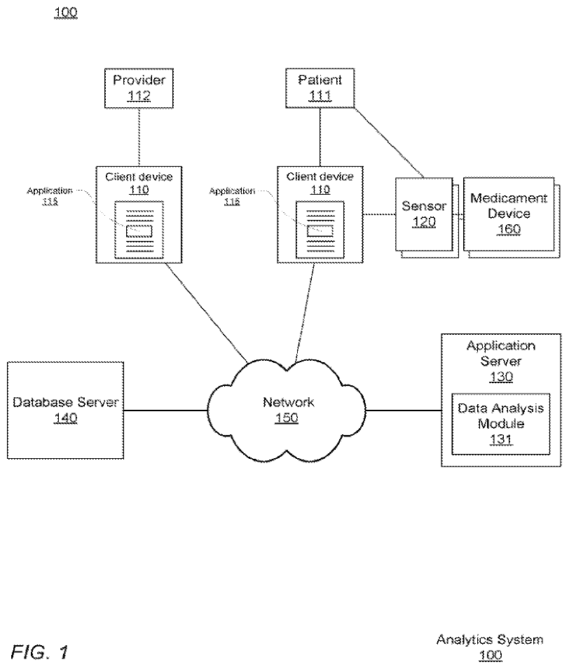

FIG. 1 shows a respiratory ailment analytics system 100 for monitoring accurate, real-time medicament device events, performing analytics on that data, and providing respiratory ailment risk notifications based on identification of triggers for a primary patient in relation to a general patient population, according to one embodiment. Such respiratory ailments may be an asthma rescue event that may be addressed through prompt treatment of medicament through an inhaler.

The respiratory ailment analytics system includes client computing devices 110, a medicament device sensor 120, a medicament device 160, an application server 130, database server 140, and a network 150. Although FIG. 1 illustrates only a single instance of most of the components of the respiratory ailment analytics system 100, in practice more than one of each component may be present, and additional or fewer components may be used.

The client devices 110, at the behest of their users, interact with the respiratory ailment analytics system 100 via the network 150. For purposes of explanation and clarity it is useful to identify at least two different types of users. A patient 111 is a user burdened with asthma who makes use of the respiratory ailment analytics system 100 at least in part to obtain personalized asthma rescue event risk notifications provided by the server 130 and asthma management notifications created by their health care provider 112 in this example. Such notifications can be provided in exchange for the user's permission to allow the respiratory ailment analytics system 100 to monitor the patient's 111 medicament device 160 usage. As will be explained below, medication events are detected by a sensor 120 associated with the medicament device 160 and the user's client device 100, which in turn reports to the application server 130, which in turn can initiate a process to generate risk notifications which are provided to the user through the client device 110. In this example, the patients 111 represent a patient population, for which individual data is taken for each patient in the group. The overall statistics for the patient population are used for the trigger event analysis as described herein.

Another type of user is a healthcare provider 112 who, again with the express permission of the patient 111, also receives notifications regarding a patient's asthma management, as well as aggregated asthma community rescue event data and derived statistics regarding asthma events and other associated data. Other types of users are also contemplated, such as parents/guardians of patients 111 who may also want to receive notifications in the event that their own client devices 110 are distinct from that of their children.

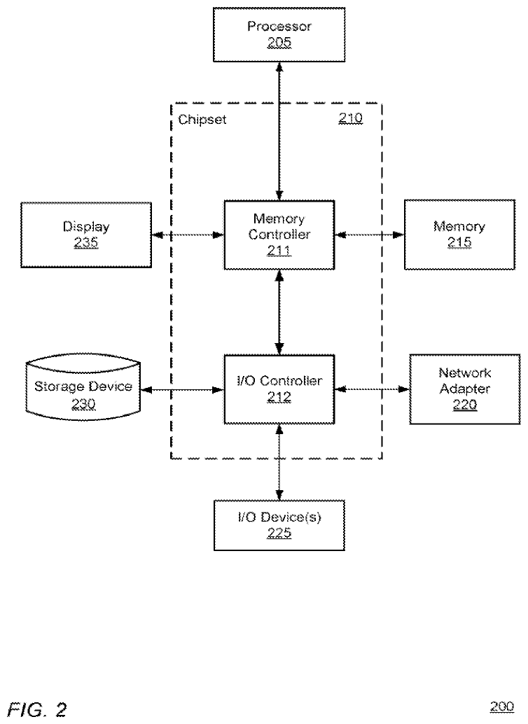

The client device 110 is a computer system. An example physical implementation is described more completely below with respect to FIG. 2. The client device 110 is configured to wirelessly communicate with the respiratory ailment analytics system 100 via network 150. With network 150 access, the client device 110 transmits to system 100 the user's geographical location and the time of a rescue medication event, as well as information describing the event as received from the associated medicament device sensor 120 (referred to throughout as “sensor 120”).

Regarding user location and event times, the client device 110 may determine the geographical location and time of a rescue event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event can be provided by the sensor 120 as part of the event data or added to event data by querying an appropriate software routine available as part of the client device's native operating system.

In addition to communicating with the application server 130, client devices 110 connected wirelessly to the respiratory ailment analytics system 100 may also exchange information with other connected client devices 110. For example, through a client software application 115, a healthcare provider 112 may receive a risk exacerbation notification describing a recent rescue event about a patient 111, then in response send a recommendation to the patient 111 for post-asthma rescue event treatment. Similarly, through application 115 patients 111 may communicate with their health care providers 112 and other patients 111.

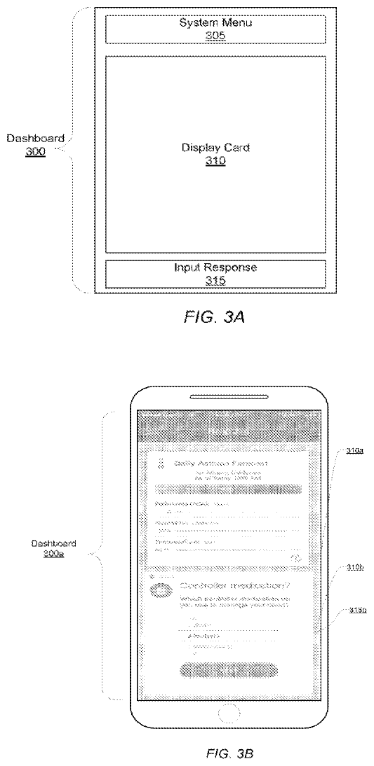

Application 115 provides a user interface (herein referred to as a “dashboard”) that is displayed on a screen of the client device 110 and allows a user to input commands to control the operation of the application 115. The dashboard is the mechanism by which healthcare providers 112 and patients 111 access the respiratory ailment analytics system 100. For example, the dashboard allows patients 111 and providers 112 to interact with each other, receive asthma rescue event risk notifications, exchange messages about treatment, provide and receive additional event and non-event data, and so on. Application 115 may be coded as a web page, series of web pages, or content otherwise coded to render within an internet browser. Application 115 may also be coded as a proprietary application configured to operate on the native operating system of the client device 110. The dashboard is more completely described below in conjunction with FIGS. 3A-3B.

In addition to providing the dashboard, application 115 may also perform some data processing on asthma rescue event data locally using the resources of client device 110 before sending the processed data through the network 150. Event data sent through the network 110 is received by the application server 130 where it is analyzed and processed for storage and retrieval in conjunction with database server 140. The application server 130 may direct retrieval and storage request to the database system 130 as required by the client application 115. As will be explained, this analysis may be extended to include event data from multiple patients 111.

The client device 110 communicates with the sensor 120 using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After the sensor 120 and client device 110 have been paired with each other using a BTLE passkey, the sensor 120 automatically synchronizes and communicates information relating to medicament device usage with the client device 110. If the sensor 120 hasn't been paired with a client device 110 prior to a rescue medication event, the information is stored locally until such a pairing occurs. Upon pairing, the sensor 120 communicates any stored event records to the client device 110. In other implementations, other types of wireless connections are used (e.g., infrared or IEEE 802.11).

Although client devices 110 and medicament devices 160 are described above as being separate physical devices (such as smart phones and inhalers, respectively), in the future it is contemplated the medicament devices 160 may include not only sensors 120 integrated into a single housing with the device 160, but also aspects of the client device 110. For example, a medicament device 160 may include an audiovisual interface including a display or other lighting elements as well as speakers for presenting visual audible information. In such an implementation the medicament device 160 itself may present the contents of notifications provided by the server 130 directly, in place of or in addition to presenting them through the client devices 110.

The medicament device 160 is a medical device used to deliver medication to the lungs of a user experiencing constricted respiratory airflow. Medicament devices (e.g. inhalers) are typically portable and small enough to be carried by hand for ease of accessibility when treating respiratory attacks. In one embodiment, medicine is delivered in aerosol form through a medicament device 160 such as a metered dose inhaler. Metered dose inhalers included a pressured, propellant canister of aerosol medicine, a metering valve for delivering a regulated medicine dosage amount, and a plastic holder that holds the pressurized canister and also forms a mouthpiece for delivery of the medicine. In another embodiment, medicine is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece to deliver dry powder to the user. Examples of controller medications that are dispensed by a controller medicament device 160 include beclomethasone, budesonide, and fluticasone as well as combinations of those medications with a long-acting bronchodilator such as salmeterol or formoterol. Examples of rescue medications that are dispensed by a rescue medicament device 160 include albuterol, salbutamol, levalbuterol, metaproterenol, and terbutaline.

Each patient may be associated with more than one medicament device 160. For example, the patient may have a rescue medicament device 160 that dispenses rescue medication, and a controller medicament device 160 that dispenses controller medication. Similarly, each patient may be associated with more than one sensor 120, each chosen to operate with one of the patient's medicament devices 160.

Generally, a sensor 120 is a physical device that monitors the usage of the medicament dispenser 160. The sensor 120 is either removably attachable to the medicament dispenser without impeding the operation of the medication dispenser, or the sensor 120 is an integrated component that is a native part of the medicament dispenser 160 as made available by its manufacturer.

The sensor 120 includes its own network adapter (not shown) that communicates with the client device 110 either through a wired connection, or more typically through a wireless radio frequency connection. In one embodiment, the network adapter is a Bluetooth Low Energy (BTLE) wireless transmitter, however in other embodiments other types of wireless communication may be used (e.g., infrared, IEEE 802.11).

The sensor 120 may also be configured to communicate more directly with the application server 130. For example, if the network adapter of the sensor 120 is configured to communicate via a wireless standard such as IEEE 802.11 or LTE, the adapter may exchange data with a wireless access point such as a wireless router, which may in turn communicate with the application server 130 without necessarily involving the client device 110 in every exchange of data. These two methods of communicating are not mutually exclusive, and the sensor 120 may be configured to communicate with both the client device 110 and the application server 130, for example using redundant transmission to ensure event data arrives at the application server 130 or to provide information directly to the client device 110 while the application server 130 is determining what notification to provide in response to an event.

As introduced above, the sensor 120 captures data about usage of the medicament device 160. Specifically, each sensor 120 captures the time and geographical location of the rescue medication event, that is, usages of the rescue medicament device 160, by the patient 111. Each sensor 120 transmits the event data in real-time or as soon as a network connection is achieved, automatically without input from the patient 111 or health care provider 112. The medication event information is sent to the application server 130 for use in analysis, generation of asthma rescue event notifications, and in aggregate analyses of event data across multiple patients.

To accomplish this goal, there are a number of different ways for the sensor 120 to be constructed, and in part the construction will depend upon the construction of the medicament device itself 160. Generally, all sensors 120 will include an onboard processor, persistent memory, and the network adapter mentioned above that together function to record, store, and report medication event information to the client device 110 and/or server 130. Sensors 120 may also include a clock for recording the time and date of events.

Regarding specific sensor 120 constructions, traditional inhalers, such as mechanical dose counters, are not designed with sensors 120 in mind, and thus the sensor 120 may be constructed accordingly. Some implementations in this manner include mechanical, electrical, or optical sensors to detect movement of the device 160, priming of the device, activation of the device, inhalation by the user, etc. In contrast, modern inhalers, such as deflectable membrane dose counters, include electrical circuitry may report event information as an electrical data signal which a sensor 120 is designed to receive and interpret, for example the medicament device 160 itself may report movement, priming, and activation to the sensor 120.

More information regarding hardware and software components for the sensors 120 and medicament devices 160, as well as the interaction between them to record rescue medication events can be found in U.S. patent application Ser. No. 12/348,424, filed Jan. 1, 2009, and International Application No. PCT/US2014/039014, filed May 21, 2014, both of which are incorporated by reference herein in their entirety.

The application server 130 is a computer or network of computers. Although a simplified example is illustrated in FIG. 2, typically the application server will be a server class system that uses powerful processors, large memory, and faster network components compared to a typical computing system used, for example, as a client device 110. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of the asthma analytics system 100 by many different client devices 110 through network 150, and thus at a high level can be generally characterized as a cloud-based system. The application server 130 generally provides a platform for patients 111 and healthcare providers 112 to report data recorded by the sensors associated with their medicament devices 160 including both rescue medication and controller medication events, collaborate on asthma treatment plans, browse and obtain information relating to their condition and geographic location, and make use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server 140 has been updated.

The application server 130 stores and manages data at least in part on a patient by patient basis. Towards this end, the application server 130 creates a patient profile for each user. The patient profile is a set of data that characterizes a patient 111 of the respiration ailment analytics system 100. The patient profile may include identity information about the patient such as age, gender, current rescue medication, current controller medication, notification preferences, a controller medication adherence plan, a relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 and their personal healthcare providers 112, as well as the frequency with which notifications are provided. For example, a patient 111 may authorize a healthcare provider 112 to receive notifications indicating a rescue event. The patient 111 may also authorize their healthcare provider 112 be given access to their patient profile and rescue event history. If the healthcare provider 112 is provided access to the patient profile of the patient 111, the healthcare provider may specify controller adherence or rescue medication plans. Medication plans may include a prescribed number of doses per day for controller medications.

The application server 130 also creates profiles for health care providers 112. A health care provider profile may include identifying information about the health care provider 112, such as the office location, qualifications and certifications, and so on. The health care provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, or yearly).

The application server 130 receives rescue medication event information from the client device 110 or the sensor 120, triggering a variety of routines on the application server 130. In the example implementations described below, the data analysis module 131 executes routines to access respiratory ailment event data as well as other data including a patient's profile, analyze the data, and output the results of its analysis to both patients 111 and providers 112. This process is generally referred to as a respiratory ailment risk analysis. In this example, the risk analysis is performed in relation to asthma for the patient 111. The asthma risk analysis may be performed at any point in time, in response to a rescue event, due to a relevant change in the patient's environment, and in response to any one of a number of triggering conditions discussed further below.

Other analyses are also possible. For example, a risk analysis may be performed on rescue and controller medication use for multiple patients to identify based on spatial/temporal clusters (or outbreaks) of medication use based on historically significant permutations from individual, geographic, clinical, epidemiologic, demographic, or spatial or temporal baselines or predicted or expected values. Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations.

Responsive to any analyses performed, the application server 130 prepares and delivers push notifications to send to patients 111, authorized healthcare providers 112, and/or other users provided access to the patient's profile. Notifications can provide details about the timing, location, and affected patient(s) 111 involved in a medication rescue event. Notifications may additionally comprise a distress or emergency signal that requests emergency assistance that are distributed to emergency assistance providers 112. Notifications may also include the results of the asthma risk analysis performed by the data analysis module 131. More information regarding the types of notifications that may be sent and the content they may contain is further described below.

In addition to providing push notifications in response to an asthma risk analysis, notifications may also be provided as pull notifications, at particular time intervals. Additionally, some notifications (whether push or pull) may be triggered not in response to an asthma risk analysis performed in response to a rescue medication event, but instead in response to a risk analysis performed in response to one of the underlying factors in the asthma risk analysis changing, such that an updated notification is warranted. For example, if weather conditions indicate that an increase in air pollution is occurring or is imminent, this may trigger the carrying out of asthma risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

The database server 140 stores patient and provider data related data such as profiles, medication events, patient medical history (e.g., electronic medical records). Patient and provider data is encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy.

The database server 140 also stores non-patient data used in asthma risk analyses. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the air quality index, and so on. Another example is georeferenced pollution data, including particulate counts for various pollutants at an instance of time or measured empirically. The regional data includes information about the current weather conditions for the time and place of the rescue event such as temperature, humidity, air quality index. All of the items of data above may vary over time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130 the database server 140 may alternatively be a hardware component that is part of another server such as server 130, such that the database server 140 is implemented as one or more persistent storage devices, with the software application layer for interfacing with the stored data in the database is a part of that other server 130.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with patients, groups of patients, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

The network 150 represents the various wired and wireless communication pathways between the client 110 devices, the sensor 120, the application server 130, and the database server 140. Network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), Secure HTTP (HTTPS) and/or virtual private networks (VPNs). In another embodiment, the entities can use custom and/or dedicated data communications technologies instead of, or in addition to, the ones described above.

FIG. 2 is a high-level block diagram illustrating physical components of an example computer 200 that may be used as part of a client device 110, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analyses introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

The dashboard, for example dashboard 300 illustrated in FIG. 3A, allows users to interact with the respiratory ailment analytics system 100. The dashboard 300 provides a means to transfer information on a user-to-user (e.g., patient 111 to provider 112) or user-to-system/system-to-user basis. Dashboards 300 are accessed through the client application 115 on the client device 110 and provide a mechanism for both patients and healthcare providers to monitor medication rescue events, exchange personalized patient healthcare information, and receive notifications such as asthma rescue event risk notifications. Patients may communicate with other health care providers and other patients through the dashboard 300, for example, to discuss and share information about asthma, medication usage, or asthma management. The ability to share asthma healthcare information may give patients or healthcare care providers experiencing a similar issue a way to share individual perspectives.

The dashboard 300 also allows authorized health care providers 112 to access a list of patients to view, annotate, update, interact with, and export information about asthma patient and community data and statistics in various demographics or geographic segments. Using the dashboard 300, healthcare providers are able to monitor patients individually or in aggregate, to receive and provide feedback on how their associated patient populations are responding to asthma management guidance. A healthcare provider who has access to individual or multiple patients has the ability to establish notification thresholds, set parameters for the notifications, and receive notifications when patients' event history matches certain conditions (e.g. rescue event). Additionally, the dashboard 300 can receive and display regular reports of event patterns for specific demographics generated by the asthma analytics system 100 in relation to the general patient population as will be explained below.

The dashboard 300 presents a variety of information including tabular data, graphical visualizations, and analyses to users through display “cards” 310. Display cards 310 are conformably suited to smaller displays typical of portable client devices 110, for example mobile phones or tablets, and include “bite size” pieces of information that mimic the simplistic organizational style found in baseball cards. The dashboard 300 may also include a system menu 305 that allows users to navigate through different categories of healthcare information.

Notifications provided by the application server 130 are related to the display cards 310. Generally, notifications include not only information to be presented to the user through the application 115, but also parameters for specifying which display card 310 is to be used to display the contents of the notification. Any information pushed/pulled from the application server 130 may be associated with one or more cards. For example, a notification can be pushed to the patient based on the outcome of an asthma risk analysis. The dashboard 300 will process the notification and determine which card/s to use to present the information in the notification. Continuing the example, the recipient of the notification may make a request to pull data from the application server 130. The application server 130 provides the requested data in another notification, and the dashboard 300 then determines which display card 310 to display the requested information.

To interact with information presented, some display cards 310a include an input response 315 area. For example, in the display card 310b illustrated in FIG. 3B, a patient may scroll up or down in the input response 315 area to select a controller medication used to manage asthma or select the “next” to move to an additional display card 310.

The dashboard 300 may provide a variety of different display cards 310, which may be organized into categories. An information card type includes cards that display data. Information cards may, for example, display medication rescue events, statistics, and maps including both patient data and community data. Information cards are further sub-categorized into event, trend, education, and alert display cards.

Event cards include data relating to rescue medication events, such as a list of historical medication rescue events for a specific patient, or patient rescue event data overlaid on a geographical map for a specific provider. For example, the display card 310a illustrated in FIG. 3B is an event card that highlights patients experiencing asthma rescue events in a particular geographic area. Another event card may display an example medication usage report including a map of the location of a rescue usage event, environmental conditions at the location, and an input response area 315 for the recipient to add triggers for the rescue usage event. Another event trend card may display rescue device usage for the previous week including a total number of uses for the time period and a number of uses for each day.

A trend card includes statistical information presented using a graph or a chart designed for clear comprehension by the recipient. Examples of trend cards include plots of asthma rescue events over various time periods, time of day trends, days of week trends, and trigger trends. In this example, the trend card allows the primary patient to understand specific respiratory data in the context of data from a general patient population.

An education card includes information meant to educate the recipient. Education cards provide general disease information and tips for patients to reduce their risk of rescue events. Some education cards may require an input response 315 to allow recipients to specify whether the information provided is relevant or interesting for use in serving future cards.

Alert cards notify users of important information including informing a recipient that they are at risk for an event and/or that data has not been received from a device over a recent time period. Other alerts may include an alert that a setting on the client device is preventing data syncing (e.g. Bluetooth is turned off) or that a patient's asthma risk score has changed.

A survey card type solicits a user response by presenting yes/no, multiple choice, or open-ended questions for the user to respond to. For example, a healthcare provider or the asthma analytics system 100 may send a survey card with an asthma-related questionnaire to a patient 111 to determine a level of disease control for a specific patient. Additionally, a survey card may request the type of controller medication that the patient 111 uses to treat asthma symptoms. Generally, survey cards provide the application server 130 with data that may be missing from a patient's medical history or patient profile (as introduced above), and/or provide an update to potentially outdated information. One or more survey cards may be used to complete the patient enrollment process and create a patient profile for storage in database server 140. For example, when a patient 111 initially enrolls in the respiratory ailment analytics system 100, a push notification will be triggered by the application server 130 prompting the patient 111 to create a patient profile.

Example of survey cards may include a survey question asking whether a patient has made any emergency room visits as a result of asthma rescue events, information about the patient's controller medication, how many times the patient used their rescue medication to control an event, and what their controller medication daily schedule is. Survey cards may also ask about asthma triggers specific to a patient, such as whether pollen is a trigger. Some survey cards may ask a patient to rate their general quality of life on 5-point Likert scale, to rate their quality of sleep, to rate their ability to be active over the last 7 days, and so on. Other survey cards ask whether the patient feels better or worse than yesterday, whether the patient has had to go to an emergency room or hospital in last 12 months for a rescue event and so on.

In some instances, patient behavior or sensor-reported event information that is inconsistent with existing patient information may trigger the sending of a survey card in order to resolve ambiguity as to the patient's circumstances. For example, if the patient is experiencing a greater-than-expected count of asthma events, the survey card may request information about the type of medication the patient has currently listed on their medicament device 160, in order to verify that the correct medication is being used. Another example includes if the reported information about controller medication use indicates that the patient is only using the controller medication one time per day but their adherence plan indicates they are supposed to be taking their controller medication twice per day, system 100 could send a notification asking if the patient needs to change their adherence plan.

In some instances, patient behavior or sensor-reported event information that is inconsistent with existing patient information may trigger the sending of a survey card in order to resolve ambiguity as to the patient's circumstances. For example, if the patient is experiencing a greater-than-expected count of asthma events, the survey card may request information about the type of medication the patient has currently listed on their medicament device 160, in order to verify that the correct medication is being used. As another example, if the reported information about controller medication use indicates that the patient is only using the controller medication one time per day but their adherence plan indicates they are supposed to be taking their controller medication twice per day, system 100 could send a notification asking if the patient needs to change their adherence plan.

Navigation cards represent actionable data or messages that, upon user interaction, may redirect the user to another screen or card that is part of the dashboard 300. For example, if a patient wants to share information or request specific medication plans for controller medications with a physician, a navigation card would be used to facilitate the sharing of information or enrollment in controller adherence plan. Additionally, navigation cards allow users to update information surrounding medication rescue events.

Adherence cards are designed to encourage a patient to continually use their controller medication on schedule over different periods of time. Adherence cards may indicate a “streak” or continuous run of on-time controller medication events, a better performance in aggregate even if not streak has been Additionally, a survey card may inquire as to the physical state of the patient in response to recording a significant number of rescue events within a threshold period of time of each other. Controller medication events may be represented as graphs to illustrate when the patient 111 did and did not take their controller medication on time during various periods during the day, as prescribed by their health care provider 112. Cards may also detail a daily schedule for controller medication and an indicator for displaying whether the scheduled dose has been taken. For example, a red “X” may indicate the scheduled dose has not been taken, but a green check mark or a different symbol may indicate that the scheduled dose has been taken.

Setup cards guide recipients in associating sensors with client devices 110. Setup cards may guide pairing a sensor to a client device 110 using Bluetooth, prompt the recipient to initiate the pairing process or prompt the user to select a sensor device for pairing, or notify the user that the sensor is paired.

In some embodiments, the dashboard may present a user interface. The user interface may illustrate a list of rescue events, responsive to the user's selection of the “View Timeline” input response area 315c. The list displays rescue usage events over a time period and includes details such as date, time, number of puffs, and location. Recipients may edit rescue usage events and/or add additional details by selecting the edit interaction response areas. Some interfaces may present an event summary for a rescue usage event to the user. The event summary may be presented to the user in response to the user selecting the edit interaction response area of the user interface. From the dashboard, the user may also view and edit a medication list, detailing information such as medication type (rescue, controller, etc.), dosage schedule, and sensors.

To receive asthma risk notifications, a patient interfaces with the dashboard 300 to initialize a patient profile. Once the patient completes their patient profile, the client device 110 transmits the patient profile for use by the application server 130 and storage by the database server 140. Once a patient's patient profile is initialized, the application server 130 may begin to receive rescue medication events detected by the sensor 120 associated with the patient's medicament device 160. This initialization process for the patient profile is only performed once during the patient's first use of the medicament device. As will be explained below, notifications may also be generated automatically when conditions for which the patient is particularly vulnerable to become discovered in comparison to a general population of patients.

Upon the sensor detecting a rescue usage event, the patient device 111 collects and sends the rescue event data to the application server 130, where the event information is stored 415. In some embodiments, this detection and storage process is performed repeatedly at any detection of a rescue usage event. However, this frequency may differ from the frequency with which a risk analysis is performed.

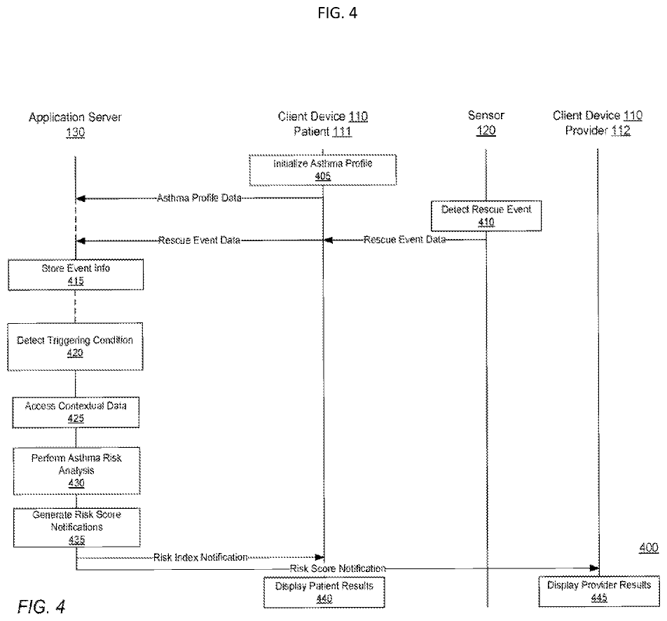

FIG. 4 is an interactive diagram for the example process for providing asthma risk notifications based on medicament device monitoring. As will be explained, the collection of data from medicament device monitoring provides large population based event warnings to patients such as the patient 111 in FIG. 1. As an initial step, a patient interfaces with the dashboard 300 in FIG. 3A to initialize 405 a patient profile. Once the patient completes their patient profile, the client device 110 transmits the patient profile for use by the application server 130 and storage by the database server 140. Once the patient profile is initialized 405, the application server 130 may receive rescue medications events detected by the sensor 120 associated with the medicament device 160 of the patient. The initializing and completing of the patient profile is only performed once during the first use of the medicament device 160.

The application server 130 generally receives a rescue event anytime the patient uses their rescue medicament dispenser 160 to relieve respiratory ailments such as asthma-related event symptoms. As an example of the process for capturing such an event for a particular device 160/sensor 120 combination, at the start of symptoms, the sensor 120 may detect whether a medication dispenser 160 cover is opened. When the medication dispenser cover is opened, the sensor 120 may detect an acceleration associated with a priming of the dispenser 160. For some types of medicament dispensers, the “priming” includes activating a mechanism to release a dose of a medication from a packaging. In other types of medicament dispensers, the “priming” includes a rapid shaking of a medication canister.

After the priming action is detected, the sensor 120 is configured to store 415 data associated with the rescue event in active memory of the sensor 120. The rescue event data may include information that describes the time and date of associated with the rescue event, the status or condition of the medicament device 160 (e.g. battery level), the number of doses of medication remaining (before or after the event), self-test results, and physiological data of a patient being treated with the medicament device 160 as measured by the sensor 120. As soon as the sensor establishes a network connection with either the client device 110 or network 150, the sensor transmits any locally stored rescue event data to the client device 110 or the application server 130. If the event data was transmitted to the client device 110 first, the client device 110 then transmits the rescue event data to the application server 130 as soon as the client device 110 establishes a network connection with the network 150. Depending upon the implementation, either the client device 110 or sensor 120 will add the geographic location where the event took place to the event data transmitted to the application server 130.

Upon receiving and storing the rescue usage event data, the application server 130 may request further information from the patient describing the rescue usage event. To obtain the information, the application server 130 generates a push notification, including the questions to be asked, to be sent to the patient's client device 110. The client device 110 may present the push notification as a survey type card 310. The patient may respond to the request by providing inputs 315 in response to the survey card 310. Alternatively, the patient 111 may elect not to respond to the request. This is permissible, any gaps in information may be obtained through later push notifications, or upon entry by provider 112 after a meeting with the patient 111. In one implementation, the failure to receive the additionally requested data in response to request does not hold up the remainder of the analysis in steps 425-445.

Based on the information collected as part of the event or otherwise, the asthma analytics system detects 420 a triggering condition. Triggering conditions refer to information pertaining to parameters that may have played a role in triggering the event, a location of the rescue event, a label (e.g., work, home, or school) for the location, a rating to signify the personal importance of the location to the patient, and whether the use was pre-emptive (e.g., medication taken prior to exercising) in addition to any other relevant information.

In addition to requesting additional event data, the application server 130 accesses 425 stored contextual data from the database server 140. Generally, contextual data refers data other than event data, which includes but is not limited to: to atmospheric conditions, weather conditions, air quality conditions, pollen data, patient data recorded from past rescue usage events, and any other considerations that are not directly detected by the medicament device at the time of the rescue usage event. By contrast, event data refers to any parameters related to the rescue event and reported by the medicament device, such as medication dosage, the time of the event, the location of the event, and relevant adherence data. Both forms of data may include temporally-current information as well as stored historical data. Accordingly, as part of obtaining the contextual data, the application server 130 also accesses historical rescue usage event data for the patient 111. This historical data can include all of the data from any past controller or rescue medication event data from the patient's history over a variety of windows of time in the past, and each historical event may include all of the same items of information as was reported 410 for this event along with any data collected upon follow up thereafter.

Once the triggering condition data is collected and a triggering event is determined 420 and the contextual data 425 is obtained, the routine performs an asthma risk analysis 430. As will be explained, this risk analysis 430 is performed in relation to both a primary patient as well as the general population of patients for each of certain selected parameters taken from the collected contextual data. As will be explained below, certain selected contextual data parameters are designated as triggering conditions based on analysis of the contextual data and the triggering events for both the primary patient and the general population of patients. In this example, the triggering events relate to specific environmental data in the contextual data. The routine then generates risk score notifications 435. The risk score notifications are generated in relation to the average risk for the environmental parameter as will be explained. The routine then sends the risk index notification to the application on the client device 110 to display patient results as shown in FIG. 3B 440. The risk index may be the percentile of the coefficient for that condition. For example, the risk index may be related to a bucket for an environmental variable as will be explained below. The routine also sends the risk score notification to the client device 110 for the provider 112 445. The risk score may also be sent to the patient 111.

However, in some instances contextual data and historical data are represented separately. Contextual data may be used to refer to geographic and regional information relevant to the current event or current location of the patient's client device 110, whereas historical data may be used to refer to geographic, regional, and event information from previous rescue usage events from the same or different patients.

When considered individually, asthma rescue inhaler usage events provide little insight into the specific causes of the rescue event, however, these events include data that is useful in identifying correlations between rescue events. For example, if only a single rescue event is analyzed, the asthma event contextual environmental data may not conclusively indicate what specific conditions caused the rescue event. However, that single event does at least indicate that certain conditions were present during the event. When the contextual data for an individual event is considered in addition to the contextual data from several other related or similar events from a general patient population, the data analysis module 131 may identify, with a high level of confidence, the triggers that a primary patient is sensitive to and not sensitive to. These trigger sensitivity scores are continuously refined by regression analysis as additional data is collected in relation to the primary patient and the patient population in general. The trigger sensitivity scores are made in relation to the distribution of raw sensitivity scores of the patient population and calculated as a percentile.

During normal course, the database 140 receives data regarding rescue usage events as they occur and updates the primary patient data store and the general patient population data store to reflect the contextual data associated with the recent current rescue usage events for the primary patient and all the patients in the patient population. The frequency with which a data store is updated with new rescue usage events may vary depending on a number of factors including but not limited to the patient's circumstances, their adherence regimen to a controller medication, environmental conditions, and so on. A patient's adherence to a prescribed controller medication regimen is a comparison between the frequency per day that the patient uses the controller inhaler unit and the frequency per day that the patient was instructed to use the controller inhaler unit and may be measured based on the usage of a controller inhaler unit.

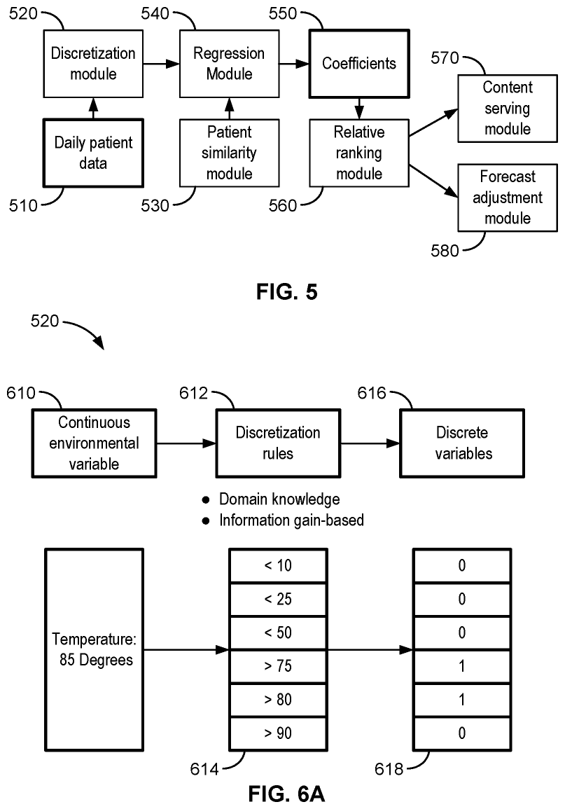

In one example, the process for determining triggers for a patient is carried out by the data analysis module 131 of the analytic system 100 in FIG. 1 based on analysis of primary patient contextual data in comparison with the triggering data from a patient population. FIG. 5 is a block diagram illustrating the logical components that carry out the functions of the data analysis module 131, according to one example. The analytics system 100 includes, on the application server 130, a data analysis module 131 that analyses the variety of data collected by the system, introduced above and discussed further below, to identify triggers for a primary patient at risk of experiencing rescue inhaler usage events. These trigger analyses are used to generate notifications that are specifically configured to be sent to the primary patient in a sufficiently timely manner to hopefully avoid or anticipate the occurrence of a respiratory event, such as an asthma or COPD event, which would necessitate usage of their rescue inhaler.

The data analysis engine 131 includes an input from the database 140 in the form of daily patient usage data 510. As will be explained below, the daily patient usage data 510 may include a primary patient data store and a secondary patient data store. The data analysis engine 131 includes a discretization module 520, a patient similarity module 530, a regression module 540, a relative ranking module 560, a content serving module 570, and a forecast adjustment module 580. Different coefficients 550 are output by the regression module 540 to evaluate each trigger condition for the primary patient.

The daily patient data 510 is the collected usage and contextual data from both a primary patient and the patient population such as patients 111 in FIG. 1. The data may include usage data from treatment devices such as the medicament device 160 in FIG. 1. Contextual data may be obtained from an independent database or other sensors. Additional relevant data such as weather, pollutants, land use, other environmental data, and activity data may be collected from other patient sensors or other sources. As explained above, the collected data is stored in a database such as database 140 in FIG. 1.

The analysis of patient-specific triggers for rescue inhaler usage events by the data analysis engine 131 is based on patient data for a population of patients, for example contextual data, demographic data, and clinical data. As described herein, the patient in question for whom the module 131 is determining triggers is referred to as the “primary patient” and each other patient of a set of similar patients is referred to as a “secondary patient.” The primary patient and the secondary patients are all members of the general patient population. The primary patient data store of the database 140 includes contextual information describing every day that the patient has been associated with the rescue inhaler such as the medicament device 160 in FIG. 1. The primary patient data also includes demographic and clinical data describing the medical condition of the primary patient and their medication regimen. Similarly, the secondary patient data store of the database 140 stores demographic, clinical data, and contextual data for the other patients of the set. The set of similar patients may be determined according to a number of methods. As one example, using the set of other patients may be determined by those patients who use a rescue inhaler connected over a same network 150 (e.g., 3G cell phone tower router, local internet service provider, etc., thus grouping patients geographically. For example, if one hundred patients use rescue inhalers communicating with the network 150, the primary patient store stores data associated with one user (for whom triggers are being determined at that instant) and the secondary patient data store stores data associated with the remaining ninety-nine. Other methods may use other criteria, such as based on contextual data, demographic data, or clinical data, specific examples of which are provided below. Primary patient data and secondary patient data generally include the same information, including contextual data, demographic data, and patient data.

Contextual data describes days during which one or more rescue events did occur and days during which no rescue usage events occurred. The primary patient data store in the database 140 may also include a record of the user's behavior over the first one month in which they are using the sensor 120 with the medicament device 160. Contextual data includes, but is not limited to, air pollutant conditions (e.g., ozone molecules (O3), nitrogen dioxide molecules (NO2), sulfur dioxide molecules (SO2), particulate matter, 2.5 micrometers or less (PM2.5), particulate matter, 10 micrometer or less (PM10), and air quality index) and weather condition (e.g., temperature, humidity, wind speed, wind direction, station pressure, and visibility). Each contextual condition also represents a potential trigger condition which contributes to or stimulates a rescue inhaler usage event. Generally, the contextual data may be gathered automatically based on device or other third party data reporting, manually provided by a patient and/or provider, or otherwise obtained. Rescue inhaler usage events and other contextual data may also be identified based on where the event temporally occurred while the sensor 120, client device 110, and/or medicament device 160 were physically located within the boundary of a geographic region.

Demographic data describes each patient including, but not limited to the gender, age, and the base location of each user. The base location refers to a geographic region within which the patient spends a majority of their time, for example a home address or an office address. The size of the geographic region may range depending on the frequency of a patient's rescue inhaler usage and their level of risk, for example 500 feet areas, latitude/longitude coordinates, zip codes, city boundaries, etc. Demographic data may be entered manually by the patient through the GUI 300 when setting up use of the sensor 120 or provided to a healthcare provider for association with their system.

Patient data may also include clinical information, for example, the medication type, the prescription amount, the prescription data, the dosage of medication taken, and the adherence regimen comparing the frequency per day with which the patient takes the rescue medication to the frequency with which the prescription is instructed to take the rescue medication. Patient data is generally entered by the healthcare provider, but may also be entered by the patient manually.

Individual primary patients may be more susceptible to specific trigger conditions than other patients. As described herein, a trigger refers to a measurable quantity, for example an environmental condition from the contextual data, which independently or in combination with one or other triggers exacerbates a patient's condition thereby causing a rescue inhaler usage event. For example, under conditions with higher levels of humidity, one patient may be more at risk of an asthma rescue usage event than another patient. Accordingly, the data analysis engine 131 determines which, if any, triggers are relevant to each patient, as well as a relative risk to the patient for each trigger in relation to the distribution among a general patient population. Thus, a coefficient of the trigger represents the absolute risk of the trigger for the primary patient. A relative risk is also determined, which is the coefficient as a quantile of the distribution of all patients.

In this example, the discretization module 520 converts continuous environmental contextual variable data into discrete variable buckets. This process groups environmental data from each type of regional area into different buckets. In this example, a region is patients in a country for convenience of types of data collected, data formats, and other factors. The buckets may be created by different techniques such as creating equal sized buckets over a range of parameter values, or creating certain peak thresholds. Peak thresholds may be determined from domain knowledge, or as a percentile of the variable experienced by patients over all. For example, the 90th percentile or above may be chosen as a bucket for ozone, and that may fall at a value of 0.07 ppm. This makes it easier to compare regressions in multiple regions with different environmental conditions, and focus on the impact of extreme conditions to determine whether the primary patient is susceptible to a trigger condition. Other data may be used to determine susceptibility. For example, land use may be analyzed as well as other location data such as the building environment related to the patient. The activities performed by the patient may also be used from worn health monitors.

For example, keeping variables continuous, a very positive coefficient for someone living in southern California may be obtained in relation to temperature triggering events. However, the temperature may only vary between 65-75 degrees in that region. This is difficult to compare an obtained coefficient to a coefficient for someone in New York City, where temperature may vary between 30 and 100 degrees. The process of discretizing variables addresses this issue, such that if a patient is not exposed to an extreme condition the discrete variable column representing that condition will be all 0s and the variable will be effectively excluded from the regression, producing a coefficient of 0. Only users who have experienced a certain triggering condition will produce a coefficient representing the risk associated with that condition, allowing a like-for-like comparison with other patients who have experienced that condition. This is not possible when variables are continuous.

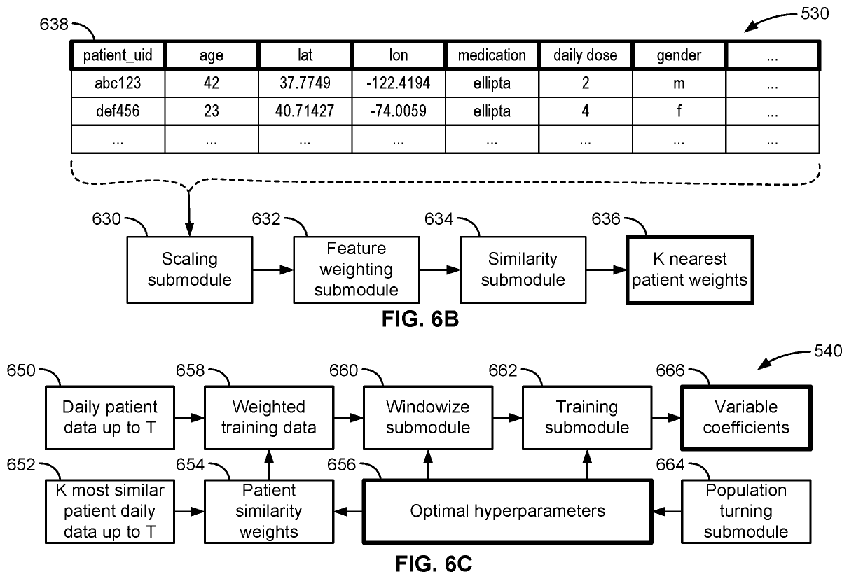

FIG. 6A shows a diagram of a process of discretization of an environmental parameter such as temperature by the discretization module 520. An example continuous environmental variable 610 such as a temperature of 85 degrees is determined from the collected daily patient data. A set of discretization rules 612 is applied to create the buckets. In this example, a discretization rule relating to temperature provides a set of temperature ranges 614. A discrete variables routine 616 classifies the obtained temperature data (85 degrees) according to the applicable rule. In this example, the routine 616 creates a series of buckets 618 that classify the obtained data. For example, the temperature data of 85 degrees is added to the buckets of greater than 75 degrees and 80 degrees via a one, while other buckets have a zero indicating the data is not classified in those buckets.

The patient similarity module 530 takes static characteristics of a patient when they first join the system, and identify the most similar patients based on those characteristics. Variables may be scaled and weighted, and similarity measures may include a Gaussian radial basis function, inverse Euclidean distance, cosine distance, or any other appropriate kernel or similarity measure. In some examples, in which sufficient primary patient data has been collected, an aggregate dataset comprised solely of primary patient data may be used. Sufficient primary patient data allows the data analysis module 131 to identify the primary patient as sensitive or insensitive to each trigger condition with a high level of confidence based only on that patient's data. However, identifications made with a high level of confidence require large amounts of data which would require primary patients to have been using their rescue inhaler usage for an extended period of time, for example multiple months to years. Accordingly, to expedite the time required for the data analysis module 131 to confidently identify trigger conditions for relatively new patients, the patient similarity module 530 supplies data from secondary patients who are demographically, contextually, and clinically similar to the primary patient to supplement the aggregate dataset in order to make these determinations with higher confidence. Thus, in these instances aggregate datasets including data from both the primary patient and secondary patients are used. The use of data from secondary patients may be necessary when data is first collected from a primary patient. Secondary patient data may be used less as more data is collected from the primary patient. In general, secondary patient data is used if there are “similar enough” patients to the primary patient. The weighting of secondary patient data is generally scaled back as more data is collected from the primary patient.

FIG. 6B shows a detailed diagram of the patient similarity module 530. The patient similarity module 540 includes a scaling submodule 630, a feature weighting submodule 632, and a similarity submodule 634. The similarity submodule 634 outputs the K nearest patient weights 636 which constitute the secondary patients that are most similar to the primary patient. A set of patient records 638 is input to the scaling submodule 630. The patient records include different types of data. In this example, the data may include a patient ID, gender, geographic location, medication, and dosage. Each of the different types of data are variables that are scaled by the scaling submodule 630. A set of weights is applied to each of the different types of data by the feature weighting submodule 632. The similarity submodule 634 determines similarities from the secondary based on the weighting. The output of the similarity submodule 634 is the K nearest patient weights, which is a number of datasets of secondary patients that are determined to be most similar to the primary patient. These datasets of K similar secondary patients are stored in the secondary patient data store and then aggregated with the data from the primary patient for input in to the regression module 540 in FIG. 5. As explained above, secondary patient data may be used when there is insufficient data from the primary patient.

The regression module 540 performs regression analysis of the data collected from a primary patient over a set period of time to provide triggering conditions for the primary patient. Additional regression analysis is performed by the regression module 540 at periodic intervals as more data is collected thus increasing the accuracy of the analysis in relation to triggering conditions. The regression module 540 performs a statistical analysis of a risk score of the primary patient in relation to each of the triggering conditions. More specifically, the regression module 540 may determine the significance, confidence, and magnitude of the risk score of the triggering condition.

FIG. 6C shows a block diagram of the components of the regression module 540. The regression module 530 includes a primary patient input 650 and a secondary patient input 652. The primary patient input 650 is daily data collected from a primary patient up to a designated time, T. The secondary patient input 652 is data collected from the patients determined to be sufficiently similar to the primary patient from the patient similarity module 530. A patient similarity weights module 654 applies patient similarity weighting 636 from the patient similarity module 530 in FIG. 6B and the optimal hyperparameters 656 to the data from the secondary patient input 652. The data from the primary patient 650 and weighted data from the secondary patients 654 are input into a weighted training data module 658. The weighted training data module 658 provides weights to each type of data collected from the patient data inputs 650 and 652 to a windowization module 660. The windowization submodule 660 provides limits to each of the types of data from the weighted training data module 658 to a training submodule 662. The windowization may include time parameters, the present condition, or the past condition for the type of data. For example, the windowizing may be used to calculate the current ozone level, or the mean and maximum ozone level of the past 2, 3, or 4 days. The optimal hyperparameters 656 are derived from the output of a population tuning submodule 664. For example, hyperparameters 656 may include the number of similar patients to include, the weighting scheme used, the size of the “lookback” window, and the regularization/penalty size used during regression. The optimal hyperparameters 656 are provided to the weights module 654, the windowization submodule 660 and the training submodule 662. The training submodule 662 outputs variable coefficients 666. The variable coefficients 666 are the relative value for each of the environmental parameters for the specific primary patient that may trigger a respiratory event.

The population tuning submodule 664 searches for the optimal hyperparameters 656 in this example. These hyperparameters may include, but are not limited to the trailing window size for variable aggregation/summary, the number of similar patients (if any), the sample weighting scheme for those similar patients (if any), and hyperparameters of the model used in the training submodule 662 itself, such as regularization/penalty terms. Optimal hyperparameters are the “settings” of the entire process that result in the minimum average error for out-of-sample data during the cross-validation process. Such hyperparameters may be optimized by training different models on a percentage of data sets to determine suitability for data not used in the training process. For example, data from the past 2, 3 or 4 days may be analyzed to determine whether 2, 3 or 4 days is the optimal hyperparameter.

The population tuning submodule 664 may identify optimal hyperparameters in different ways depending on the type of hyperparameter. For example, the optimal hyperparameter for regression-based hyperparameters, such as the trailing window size and regularization, may be identified by a process known as time series k-fold cross validation. This involves partitioning a time series of each individual into different sequential partitions of time, training individual patient models on the first partition and evaluating performance on the second partition, training on the first and second partitions and evaluating performance on the third partition. This process is repeated until the last partition, at which point the out-of-sample performance is averaged across all time partitions for all users. The hyperparameters that result in the lowest average out-of-sample error are chosen. For secondary patient identification hyperparameters, such as how many similar patients to include and the weighting scheme used for those similar patients, these may be identified by sampling different numbers of similar patients and different weighting schemes, training a single model across the resulting secondary patient data set, and then evaluating model performance on primary patient data, which is by definition out-of-sample of the secondary patient data. This is repeated for each patient and the hyperparameters that produce the lowest average error across the entire population are chosen.

The training submodule 662 runs regression analysis on the (weighted) training data from the weighted training module 658 to produce the variable coefficients 666 for the primary patient. The training submodule 662 controls for individual patient risk, seasonality and time, to isolate the impact of environmental variables for each patient over a cumulative period.

The population tuning submodule 664 may be run at a cadence significantly less than the training submodule 662 to conserve computational resources. For example, the training submodule 662 may be run every 30 days for the data collected from each primary patient, but the population tuning submodule 664 may be run over the data from the entire population of patients once every 90 days to optimize the hyperparameters incorporating the new data added.

The outputted variable coefficients 666 may be a point estimate or a distribution based on frequentist standard errors, Bayesian methods, or bootstrap methods. For example, the coefficient for temperature below 50 may be a point estimate of 0.2, or a normal distribution (produced from any of the methods referenced) with a mean of 0.3 and a standard deviation of 0.1.

The relative ranking module 560 in FIG. 5 outputs a relative ranking of the coefficient for each of the parameters for the primary patient in relation to the distribution of coefficients among the general patient population. FIG. 6D shows the components of the relative ranking module 560. The relative ranking module 550 accepts coefficients 670 for each of the environmental parameters from the regression module 540. The coefficients each relate to a variable (V) up to time T. For example, a variable may be the coefficient of temperatures below 50 for the first 30 days of every patient in the population. A ranking submodule 672 takes a point estimate or distribution of each coefficient calculated by the regression module 540 and ranks the coefficient value as a percentile 674 relative to the rest of the historical population of patients for the example variable V up to time T. A coefficient database 676 supplies the corresponding coefficient value distributions for the entire patient population to the ranking submodule 672. The percentile or percentile range 674 determined by the ranking submodule 672 is then passed into a threshold submodule 678. Threshold values may vary depending on clinical and product relevance. The threshold submodule 678 produces a label output 680 out of different label options that may include, but are not limited to, “not above average,” “above average,” or “significantly above average” based on the comparison of the coefficient of the primary patient to the threshold values of the coefficient distribution of the entire patient population for the example variable V up to time T. Of course other descriptive labels such as poor, fair and good may be used. Although three tiers of descriptive labels are used in this example, greater or less tiers of descriptive labels and corresponding ranges may be used.

The content serving module 570 provides the output labels 680 from the relative ranking module 560 to patient accessible applications such as that described in FIG. 3A. The label may be used throughout the application to produce content relating to informing the patient of their relative sensitivity, and personalizing content such as ongoing tracking and warnings of when conditions to which they are in upper percentiles of the distribution of sensitivity values.

The content serving module 570 may also compare variable labels from the current cumulative time period to previous shorter ones, to identify changes in relative sensitivity. Other content may also be served when a sensitivity label changes.

The label data 680 is also output to the forecast adjustment module 580 that may incorporate the labels in characterizing the entire risk value reflecting all of the relevant triggering conditions or events for the primary patient. The relative sensitivity labels may be used to adjust the personalized forecast for the primary patient. For example, if the conditions to which a patient is in a high percentile of sensitivity are forecast, the personalized forecast may display the more severe of the personalized forecast output and the term “fair.” For example, if the primary patient is in a higher percentile of the patient population distribution for sensitivity to temperature, the more conservative, and higher risk term “fair” may be selected for display on the application interface in FIG. 3A for the primary patient, even if the term “good” would generally be used. Further, if the conditions to which a patient has significantly above average sensitivity are forecast, the personalized forecast may display the label “poor” regardless of the underlying risk score between 0-1. In this manner, a patient may understand the degree of risk of a respiratory event or ailment in a non-statistical context.

The comparison of the coefficient for the primary patient to the distribution of coefficient values for the general patient population may be used to adjust different functions of the application in FIGS. 3A-3B. For example, an alert may be generated to the patient that a new trigger has been discovered. For example, such an alert would not be generated for most patients, but the above routine may determine that the primary patient has a high susceptibility to a certain parameter and thus for the primary patient, the alert would be generated.

Once a patient has been determined to be sensitive to a trigger by the data analysis module 131, a notification may be generated. The notification may include a list of labeled triggers, a list of triggers still being analyzed, information characterizing the trigger, the relative risk score and a comparison of the trigger in relation to the trigger occurrence for the general patient population, and options that the patient may take to prevent the occurrence of another rescue usage event in the presence of the trigger. The notification may be in the form of a card delivered through the dashboard 300 as discussed above to the client device 110. The notification may also be provided to other devices such as the healthcare provider's client device 110.

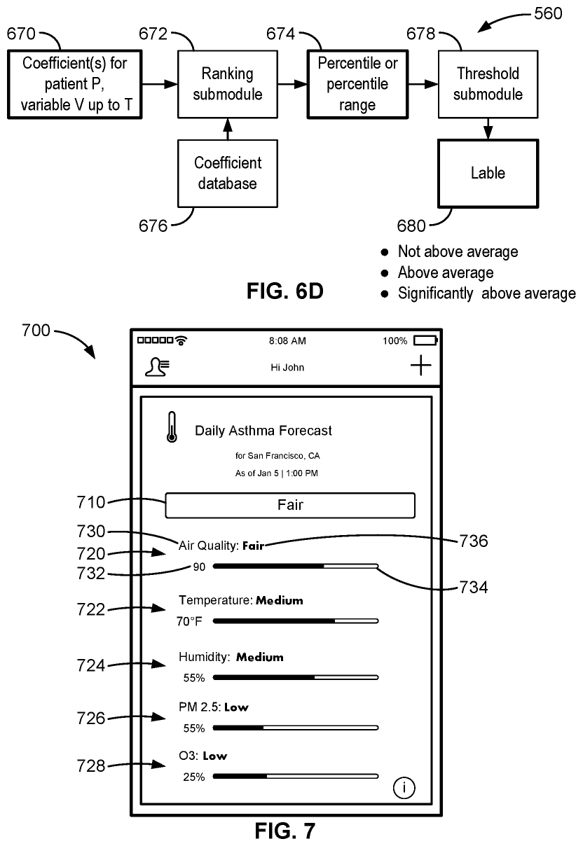

FIG. 7 shows an interface 700 that shows the variables based on the labels tailored to a primary patient as determined by the forecast adjustment module 622. The interface 700 may be generated by an application on a mobile device operated by the primary patient. The interface 700 includes an overall likelihood for trigger for respiratory condition assessment 710 based on relevant contextual parameter values for the day. As explained above, the overall likelihood is displayed as a label corresponding to the risk factor for the primary patient for the respiratory ailment such as asthma.

In addition, the interface 700 includes specific contextual parameters that are selected based on higher than expected coefficient for the primary patient. In this example, the contextual parameters of air quality, temperature, humidity, particulate matter, 2.5 micrometers or less (PM), and ozone (O3) have been analyzed as high risk parameters for the primary patient. Thus, the display 700 includes an air quality field 720, a temperature field 722, a humidity field 724, a PM 2.5 field 726, and an ozone field 728. Each of the fields such as the air quality field 720 include a description 730 of the parameter (e.g., air quality), a numerical value 732, a graph 734 showing the relative amount of the numerical value, and a descriptive label 736. The fields 720-728 are selected for the primary patient based on high percentile of the patient coefficient compared to the distribution of the coefficients population as explained above.

The distribution of coefficient values is determined from a total population of patients with potential respiratory ailments that use devices collecting usage data and have the similar time period for collection of data for the regression analysis. As explained above, after the first T days, a regression is run, which generates coefficients. Each coefficient is compared to all patients in the population who have reached at least day T, but that distribution of coefficients is only generated from regressions on the data of all of those patients up until day T. Thus, even if the comparison patient population has been using devices significantly longer than the primary patient, comparisons are made only to the exact same time period as the primary patient.

However, the general population of patients may be narrowed to a smaller subset in the above described procedures. For example, the general population of patients may be determined by filtering the total population of all patients by factors such as the medicament used, initial disease severity, or only patients experiencing a certain respiratory condition. For example, the initial disease severity for a respiratory disorder may be measured by an Asthma Control Test (ACT) score or a COPD Assessment (CAT) score). The general population may also be narrowed to exclude any patient with a 0 coefficient for that specific environmental condition, or by using any other indicator that identifies a patient has not experienced that environmental condition within the time period T.

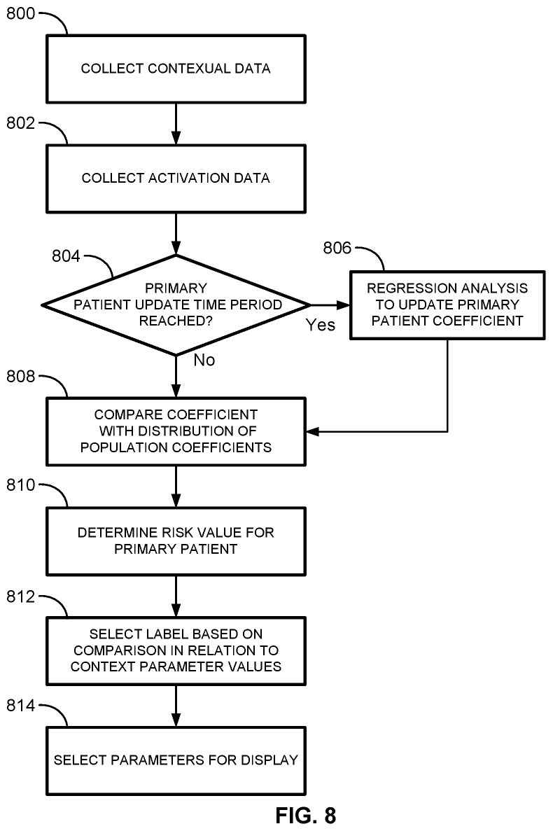

The flow diagram in FIG. 8 is representative of example machine readable instructions for collecting and analyzing data to label events based on primary patient data in relation to a general population. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIG. 8, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine in FIG. 8 is executed by the data analysis module 131. The data analysis module 131 collects contextual data relating to all patients in the patient population (800). The data analysis module 131 also collects activation data based on the determination of using a medicament dispensing device such as an inhaler (802). The routine determines whether the update time period for the primary patient has been reached (804). The regression analysis time period for the primary patient in this example is every 30 days of collected data. If the time period for the primary patient has been reached, a regression analysis on all collected data is performed to update the coefficient for the primary patient in relation to the triggering event (806). The regression analysis may include secondary patients similar to the primary patient based on the optimal K value as explained above.

After the coefficient for the primary patient is obtained, the coefficient of the primary patient is compared with the distribution of coefficients for the entire patient population (808). The routine then determines the overall risk factor for the primary patient (810). The routine then selects a label based on risk factor of the primary patient and adjusts the label selection based on the comparison with the distribution of coefficients for the patient population (812). The routine also selects parameters for display relating to coefficients where the primary patient is in a high percentile compared to the distribution of the coefficients of the general populations (814).

The continuous monitoring in the system in FIG. 1 may be used for a variety of respiratory disorders such as asthma, COPD, cystic fibrosis, and bronchiectasis. However, it is to be understood and appreciated that the principles described above is not to be limited to such use.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

One or more elements or aspects or steps, or any portion(s) thereof, from one or more of any of claims 1-40 below can be combined with one or more elements or aspects or steps, or any portion(s) thereof, from one or more of any of the other claims 1-40 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for determining conditions that trigger a respiratory ailment, the method comprising:
collecting data on activation of respiration medicament devices to deliver respiration medicament to patients in a population of patients;
a storing the activation data and patient contextual parameter data related to each activation event for the population of patients;
accessing the activation data and the contextual parameter data for a primary patient in the population of patients over a certain period of time;
determining the occurrence of rescue events based on the collected activation data;
determining a coefficient for at least one contextual parameter as a triggering event for the primary patient based on the correlation of the contextual parameter and the rescue events; and
providing a comparison of the coefficient for the at least one contextual parameter for the primary patient and the distributions of coefficients of the triggering event for the population of patients based on the correlation of the contextual parameter and the rescue events for the population of patients.

2. The method of claim 1, further comprising determining the coefficient partially based on the contextual parameter data and the occurrence of rescue events based on the collected activation data for at least one secondary patient similar to primary patient of the population of patients for the contextual parameter of the primary patient.

3. The method of claim 1, further comprising determining the coefficient based on regression analysis of the contextual parameter in relation to the collected activation data optimized by at least one hyperparameter; and performing the regression analysis after a first predetermined period of time for collected activation data and contextual data.

4-5. (canceled)

6. The method of claim 3, further comprising tuning the at least one hyperparameter based on the collected contextual parameter data for the population of patients over a second predetermined period of time.

7. The method of claim 1, wherein the respiratory ailment is asthma.

8. The method of claim 1, wherein the at least one contextual parameter includes one of air pollutant condition or weather condition.

9-10. (canceled)

11. The method of claim 1, further comprising:
determining multiple trigger conditions including the determined trigger condition;
assigning a coefficient for each of the multiple trigger conditions; and
comparing the coefficients to a distribution of coefficients for each of the multiple trigger conditions from the population of patients.

12-14. (canceled)

15. The method of claim 1, further comprising alerting the primary patient if the coefficient is in a high percentile of the distribution of coefficients of the population of patients.

16-19. (canceled)

20. A method to evaluate a triggering event for a respiratory ailment of a primary patient, the method comprising:
collecting usage data from activation of treatment devices from a population of patients from a communication interface;
collecting contextual parameter data corresponding to the population of patients from the communication interface;
storing the collected usage data and contextual parameter data in a storage device;
determining a coefficient for a triggering event from the contextual parameter and usage data for each patient of the population of patients;
determining a coefficient for the triggering event for the primary patient based on data relating to the contextual data and usage data for the primary patient;
providing a comparison of the coefficient of the primary patient in relation to a distribution of the coefficients for the population of patients.

21. The method of claim 20, further comprising collecting activation data for at least one secondary patient similar to primary patient of the population of patients for the contextual parameter of the primary patient, wherein the coefficient is determined partially based on the contextual parameter data and the occurrence of rescue events based on the at least one secondary patient.

22. The method of claim 20, wherein the coefficient is determined based on regression analysis of the contextual parameter in relation to the collected activation data optimized by at least one hyperparameter, wherein the regression analysis is performed after a first predetermined period of time for collected activation data and contextual data, wherein the coefficients of the population of patients are selected based on regression analysis performed after the first predetermined period of time for each patient in the population of patients, and wherein the at least one hyperparameter is tuned based on the collected contextual parameter data for the population of patients over a second predetermined period of time.

23-25. (canceled)

26. The method of claim 20, wherein the respiratory ailment is asthma.

27. The method of claim 20, wherein the at least one contextual parameter includes one of air pollutant condition or weather condition, wherein the pollutant condition is one of air quality index, ozone molecules (O3), nitrogen dioxide molecules (NO2), sulfur dioxide molecules (SO2), particulate matter, 2.5 micrometers or less (PM2.5), particulate matter, 10 micrometers or less (PM10), and wherein the weather condition is one of temperature, humidity, wind speed, wind direction, station pressure, and visibility.

28. (canceled)

29. (canceled)

30. The method of claim 20, further comprising:
determining multiple trigger conditions including the determined trigger condition;
assigning a coefficient for each of the multiple trigger conditions;
comparing the coefficients to a distribution of coefficients for each of the multiple trigger conditions from the population of patients; and
displaying selected trigger conditions from the multiple trigger conditions based on whether the coefficient for the primary patient is in a certain percentile of the distribution of the coefficients for the population of patients.

31. (canceled)

32. The method of claim 31, further comprising:
assigning a descriptive label correlated with an overall risk factor for the primary patient and
displaying the descriptive label and trigger conditions to the primary patient on a display of a mobile device.

33. (canceled)

34. (canceled)

35. The method of claim 20, further comprising alerting the primary patient if the coefficient is in a high percentile of the distribution of coefficients of the population of patients.

36. A system to determine conditions that trigger a respiratory ailment, the system comprising:
a communication interface to collect data on activation of respiration medicament devices to deliver respiration medicament to patients in a population of patients;
a storage device to store the activation data and patient contextual parameter data related to each activation event for the population of patients;
a data analysis module operable to access the activation data and the contextual parameter data for a primary patient in the population of patients over a certain period of time, determine the occurrence of rescue events based on the collected activation data, and determine a coefficient for at least one contextual parameter as a triggering event for the primary patient based on the correlation of the contextual parameter and the rescue events;
wherein the data analysis module is operable to provide a comparison of the coefficient for the at least one contextual parameter for the primary patient and the distributions of coefficients of the triggering event for the population of patients based on the correlation of the contextual parameter and the rescue events for the population of patients.

37. The system of claim 36, wherein the data analysis module is further operable to:
determine multiple trigger conditions including the determined trigger condition;
assign a coefficient for each of the multiple trigger conditions; and
compare the coefficients to a distribution of coefficients for each of the multiple trigger conditions from the population of patients.

38. The system of claim 37, further comprising an output application, the output application displaying selected trigger conditions from the multiple trigger conditions based on whether the coefficient for the primary patient is in a certain percentile of the distribution of the coefficients for the population of patients.

39. The system of claim 36, further comprising a display displaying the descriptive label to the primary patient.

40. (canceled)
